(19) United States
(12) Patent Application Publication
(10) Pub. No.: US 2025/0017762 A1
(43) Pub. Date: Jan. 16, 2025

(54) SNORING AND SLEEP APNEA PREVENTING TOOL

(71) Applicant: Young Kil CHA, Incheon (KR)

(72) Inventor: Young Kil CHA, Incheon (KR)

(21) Appl. No.: 18/579,867

(22) PCT Filed: Nov. 23, 2022
(86) PCT No.: PCT/KR2022/018581

§ 371 Date: Aug. 1, 2024

(30) Foreign Application Priority Data
Nov. 24, 2021 (KR) 10-2021-0163109

(51) Int. Cl. A61F 5/56; A61M 16/06

(52) U.S. Cl. CPC A61F 5/566; A61M 16/0605

(57) ABSTRACT

The invention is a snoring and sleep apnea preventing tool (1) to prevent snoring and sleep apnea, comprising a preventing tool body (10), an upper teeth stopper projection (20) on the upper surface of the front end portion of the preventing tool body (10), a lower teeth guide slope portion (30) on the bottom surface of the front end portion of the preventing tool body (10) and a stopper wing (50) on the preventing tool body (10).

### TECHNICAL FIELD

The present invention relates to a snoring and sleep apnea preventing tool, and more particularly to a snoring and sleep apnea preventing tool which is small in size so as to be easily stored and is easily mountable only by biting it with the user's mouth and has an enhanced safety by preventing the airway of the user's throat from blocking by the inserted tool even though the user's upper and lower teeth are opened wide.

### BACKGROUND ART

Generally, a snoring is a sound generating phenomenon by vibration of the soft palate, the uvula or the surrounding mucous membrane when the throat muscles are relaxed during sleeping, and the oral cavity is narrowed and then the air passes through them. If the snoring becomes worsen, it can incur heavy noises and a sleep apnea. The sleep apnea is a phenomenon in which tissues around the airway of the throat are loosened and block the airway momentarily to incur suspending of respiration.

This sleep disorder may deteriorate the quality of sleeping and incur a failure of memory and a hyperkinetic syndrome due to the dwindle of oxygen supply to the brain as well as the heavy drowsiness and a chronic fatigue despite of long sleeping time. If these syndromes are neglected, it can incur a high blood pressure, an arrhythmia and as much as a sudden death. And the heavy drowsiness can be a cause of important safety accident such as car accidents.

Conventionally, mouthpiece type snoring prevention devices had been suggested to cure the snoring and sleep apnea. But these devices are of the type to be inserted into the upper teeth and the lower teeth respectively and the size of the devices is so big that it cannot be easily stored, can incur the difficulty in producing with reasonable cost. And if the set of teeth of the user is not uniform, the mounting of the device into the mouth of the user is inconvenient, and considering of it, the snoring preventing device should be produced by customization to the user's set of teeth personally, which will result in the high cost of the device, so it cannot be widely supplied with reasonable cost. Therefore, the suggestion of the snoring prevention device which is easily usable, easily storable, and moderate cost is demanded.

### DISCLOSURE

### Technical Problem

The invention is proposed to solve the above-mentioned problems, and the object of the invention is to provide a snoring and sleep apnea preventing tool, which is small in size to be easily stored and easily mountable only by biting it with the user's mouth and the airway of the throat could be naturally secured without inserting and fixing it between the user's upper teeth and lower teeth, and the mouth can be easily moved during the sleep to enhance the user convenience or comfort, and even though, while sleeping, the gap between upper teeth and the lower teeth is widen, it can be prevented from being inserted into and blocking the throat airway to secure the safety, and it can be selectively used according to user's preference and adjusted to the thickness or shape of user's mouth, and structurally sturdy as both the upper teeth and the lower teeth of the user are engaged at the same time, and is shaped of oral cavity of the mouth to reduce the mental repulsion of the user, and it can not be arbitrarily separated by cough as it is inserted deeply into the oral cavity of the user, and ventilating is effectively achieved by preventing the tongue from being curled and blocking the airway of the throat.

### Technical Solution

According to an aspect of the invention, there is provided a snoring and sleep apnea preventing tool 1 to prevent snoring and sleep apnea comprising:
a preventing tool body 10 which is extended back and forth and the front end portion of which is inserted between upper teeth 2 and lower teeth 3 of a user and the rear end portion is extended backward along the tongue 6 of the user to press the tongue 6;
an upper teeth stopper projection 20 which is provided on the upper surface of the front end of the preventing tool body 10 and upwardly protrudes so as to prevent the upper teeth 2 of the user from protruding forward;
a lower teeth guide slope portion 30 which is provided on the bottom surface of the front end of the preventing tool body 10 and is upwardly inclined toward the front end so that, when the lower teeth 3 of the user bite the bottom surface of the preventing tool body 10, the lower teeth 3 can protrude forward further than the upper teeth 2 stopped by the upper teeth stopper projection 20;
a stopper wing 50 which is provided on the preventing tool body 10 and is expanded so as to be stopped by the lips 4, 5 of the user in front of the upper teeth stopper projection 20; wherein,
air paths 11, 12 of the shape of groove extending back and forth are formed on the sides of the preventing tool body 10 for the outer air to flow into the oral cavity of the user.

According to other aspect of the invention, there is provided a snoring and sleep apnea preventing tool 1 to prevent snoring and sleep apnea, wherein an insert hole 51 is penetrated on the stopper wing 50 in the back and forth direction, and the preventing tool body 10 is further includes a front inserting projection 40 extending forward from the front end of it and being releasably engaged into the insert hole 51 of the stopper wing 50, and the stopper wing 50 can be mountable on the front insert projection 40 while being moved back and forth along the front insert projection 40.

### Advantageous Effect

According to the invention, unlike the conventional one which is mounted to enclose the entire upper teeth and lower teeth respectively, comprises the upper teeth stopper projection 20 on the upper surface of the front end of the preventing tool body 10 and the lower teeth guide slope portion 30 on the bottom surface of the front end of the preventing tool body 10 to guide the lower teeth 3 to be projected toward than the upper teeth 2, if only the user bites the preventing tool body 10, the lower teeth 3 can be naturally projected forward than the upper teeth 2 to secure the airway of the throat, it can be made with simple structure, easy production, small size to be easily stored, and it can be used for more people, because even though people with irregular teeth set can also mount it easily without enclosing the entire upper teeth and lower teeth, and range of the customer can be expanded to the disadvantaged people, eventually resulting in the enhancement of user conveniences.

And as the stopper wing 50 is provided on the front end of the preventing tool body 10 to be engaged by the lips 4,5 of the user, even though the upper teeth 2 and the lower teeth 3 are widen during sleeping and the upper teeth 2 is separated from the upper teeth stopper projection 20, the lips 4,5 are still in the state of being engaged with the stopper wing 50, so the preventing tool body 10 can be prevented from being induced into the throat of the user and blocking the airway, which will enhance the safety. And, an insert hole 51 is formed on the stopper wing 50 and a front inserting projection 40 are provided on the preventing tool body 10 to be engaged into the insert hole 51 detachably and movably, so the stopper wing 50 can be selectively used according to the user's preference, and it can be adjusted along the front insert projection 50 according to the thickness and shape of the user's lips 4, 5.

And as the preventing tool body 10 of the invention is placed or inserted simply between the upper teeth 2 and the lower teeth 3 and not engaged with the upper teeth 2 and the lower teeth respectively, the user can move the lips freely during sleeping so the quality of sleep can be improved. And as shape of the preventing tool body 10 is similar with that of the oral cavity, the wearing feeling of it is good and the user can use it without mental repulsion. And the preventing tool body 10 extends back and forth to be placed deeply in the oral cavity of the user, ejecting out of the body 10 from the oral cavity due to sleep apnea or coughing during sleeping can be effectively prevented. And as the lower surface of the preventing tool body 10 presses the tongue 6, it can prevent the tongue 6 from curing up or being lifted so that the air pathway can be easily secured to increase the above effects.

And as the air path 11, 12 are formed on the shape of recess on the sides of the preventing tool body 10, the outer air can be easily flowed into the oral cavity irrespective of the mouth shapes of the user to prevent the snoring and sleep apnea effectively.

### DESCRIPTION OF THE DRAWINGS

FIG. 1 is a perspective of one embodiment of the invention.

FIG. 2 is a side view showing the used state of the embodiment of the invention.

FIG. 3 is a plan view showing the used state of the embodiment of the invention.

FIG. 4 is a front view showing the used state of the embodiment of the invention.

### DETAILED DESCRIPTION OF THE INVENTION

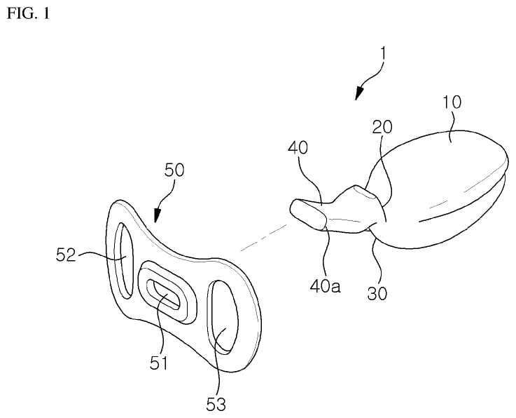

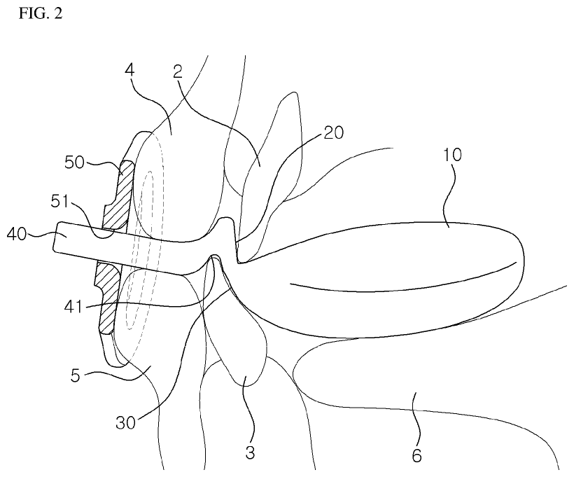

Hereinafter, the preferred embodiments of the invention will be described with reference to the drawings. FIGS. 1 to FIG. 4 show an embodiment of snoring preventing tool according to the invention. As shown, the sorning preventing tool 1 of the invention includes a preventing tool body 10, an upper teeth stopper projection 20, a lower teeth guide slope portion 30 and a stopper wing 50. The preventing tool body 10 is of the proper size which can be easily inserted on the tongue 6 and can be bitten by the upper and lower teeth 2, 3, and the front end portion of it is inserted or placed between the upper teeth 2 and the lower teeth 3, and the rear end portion extends backward along the tongue 6. As shown in FIG. 2, the preventing tool body 10 extends back and forth to be placed deeply in the oral cavity of the user, the preventing tool body 10 cannot easily ejected out of the oral cavity even though the user does the sleep apnea or coughs during sleeping.

And preferably, the shape of the preventing tool body 10 is similar with that of oral cavity, so the wearing feeling of it is good and the user can use it without mental repulse. And the thickness of the preventing tool body 10 is properly determined so that the lower surface of the preventing tool body 10 can press the tongue 6 of the user. As the preventing tool body 10 presses the tongue 6 of the user, the tongue 6 is prevented from curling up or being lifted to secure the air pathway effectively.

Meanwhile, the upper surface of the preventing tool body 10 may be convex, concave, flat or in other shape. If the upper surface of the preventing tool body 10 is convex, the preventing tool body 10 can not only be prevented from ejecting forward by the upper teeth stopper projection 20 blocking the upper teeth 2 but also be prevented from retreading backward by the convex upper surface of the preventing tool body 10 so that the preventing tool body 10 can be located on proper position. Accordingly, the preventing tool body 10 cannot be floated between the upper teeth 2 and the lower teeth 3 by motion of the upper teeth 2, and the user can stably bite the preventing tool body 10. And the shape of the preventing tool body 10 becomes more similar with that of the oral cavity, so the user can bite it with better feeling of wearing,

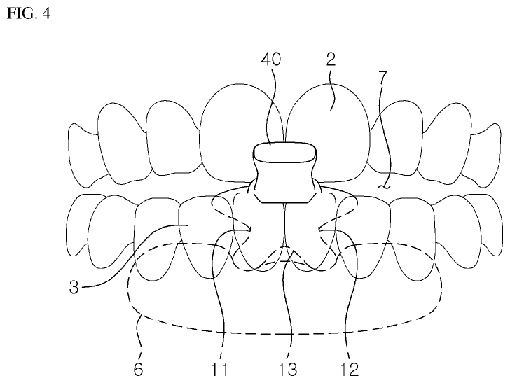

The lower surface of the preventing tool body 10 can be convex, concave, flat or in other shape. As shown in FIG. 4, if the lower surface of the preventing tool body 10 is concave to form a recess 13 extending back and forth, the contacting area between the lower surface of the preventing tool body 10 and the tongue 6 is reduced to improve the feeling of wearing, and increasing of saliva generation due to the increased contacting area can be prevented, so it can be prevented that the increased saliva blocks the air path way.

The upper teeth stopper projection 20 projects upwardly from the front end of the preventing tool body 10, so the upper teeth 2 can be blocked from extending forward. Part of the upper teeth stopper projection 20 can extend further to achieve sufficient engagement with the upper teeth 2 or upper gum.

The lower teeth guide slope portion 30 is formed on the bottom surface of the front end of the preventing tool body 10 and is upwardly inclined toward the front end of it. Accordingly, if the user bites the preventing tool body 10, the upper teeth 2 is engaged with the upper teeth stopper projection 20, and the lower teeth 3 moves forward along the lower teeth guide slope portion 30y so that the lower teeth 3 is projected forward further than the upper teeth 2.

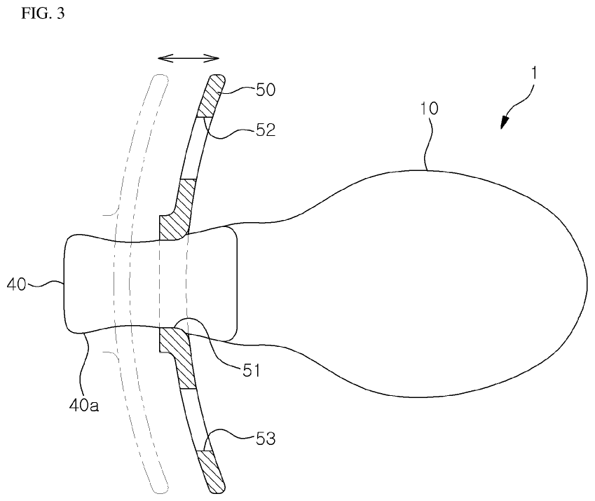

The stopper wing 50 is provided on the front of the preventing tool body 10 and extends to be engaged with the lips 4, 5 of the user. Therefore, if gap between the upper teeth 2 and the lower teeth 3 is widen during sleeping while wearing the preventing tool body 10, the preventing tool body 10 is prevented from flowing into the throat of the user becasue the upper teeth 2 are released from the upper teeth stopper projection 20. As shown in FIG. 1 and FIG. 3, the stopper wing 50 is formed round to cover the lips 4, 5 of the user when mounted in the mouth.

Openings 52, 53 may be formed on both side of the insert holes 51 of the stopper wing 50, into which a portion of the lips 4, 5 can be engaged so that the widening of the lips 4, 5 can be prevented in some degree.

According to the embodiment of the invention as described above, if the user only bites the preventing tool body 10, the upper teeth 2 is engaged with the upper teeth 20 and the lower teeth 3 moves forward along the lower teeth guide slope portion 30 and naturally projected forward further than the upper teeth 2 to achieve sufficient air pathway, so that snoring and sleep apnea can be prevented or alleviated effectively And to wear the tool, the user should only bite the preventing tool body 10 between the upper teeth 2 and the lower teeth 3, there will be no problem or inconvenience such that the tool in engaged with the upper teeth and lower teeth, so the user cannot freely move the mouth left and right during sleeping. Therefore, insomnia or frequent awakening during sleeping is prevented or alleviated to achieve improved quality of sleeping.

As shown in FIG. 4, as the bottom surface of the preventing tool body 10 presses the upper surface of the tongue of the user, the tongue is prevented from curing up or being lifted, which will effectively prevent the air pathway 7 formed between the upper teeth 2 and the lower teeth 3 extending forward further than the upper teeth 2 from being blocked or narrowed by the curled-up or lifted tongue 6.

And, as the stopper wing 50 is engaged with the user's lip 4, 5, the preventing tool body 10 is prevented from flowing into the throat of the user in case that the upper teeth 2 and the lower teeth 3 open wide so the upper teeth 2 releases from the upper teeth stopper projection 20, which will enhance the safety of the tool.

According to the invention, a front inserting projection 40 is further provided, which extends forward from the front end of preventing tool body 10 and is releasably engaged with the stopper wing 50. A lower teeth stopper projection 41 may be further provided on the lower surface of the front inserting recess 41,

As shown in FIG. 1, an insert hole 51 is provided on the middle of the stopper wing 50 into which the front inserting projection 40 of the preventing tool body 10 is engaged. Preferably, the prevention fool body 10 is made of elastic material such as rubber, and the front inserting projection 40 has the progressively enlarged front end 40a, so that if the stopper wing 50 is forcedly inserted into the insert hole 51 of the stopper wing 50, it can be readily released. As described above, the stopper wing 50 is releasably engaged with the preventing tool body 50, the user can use or may not use the stopper wing 50 depending on user's preference.

As shown in FIG. 2, even though the lower teeth 2 moves along the lower teeth guide slope portion 30 and projected forward further than the upper teeth 2, it may be engaged with the lower teeth stopper recess 41 of the front inserting projection 40, so that the lower teeth 3 is prevented from projecting further, loads against the jaw joint due to the extensive projection of the lower teeth 3 is prevented.

As shown in FIG. 3, the stopper wing 50 is mounted on the front inserting projection 40 with being moved backward and forward according to the thickness or the shape of the lips 4, 5 of the user.

According to the preferred embodiment of the invention, air paths 11, 12 are formed on the sides of the preventing body 10, As shown in FIG. 4, the air paths are of the shape of groove and extend back and forth for the outer air to flow into the oral cavity of the user rapidly. As a result, depending on the thickness of the user, even though the air pathway 7 secured by the upper teeth 2 and the lower teeth 3 projected forward further than the upper teeth 2 is closed by the lips, the outer air can flow into the oral cavity through the air paths 11, 12 to achieve a reliable effect.

The scope of the invention is not restricted by above mentioned embodiments and the drawings, and various modifications or variations will be possible by the skilled man in the art within the scope of the invention defined by the claims.

1. A snoring and sleep apnea preventing tool 1 to prevent snoring and sleep apnea comprising:
a preventing tool body 10 which is extended back and forth and the front end portion of which is inserted between upper teeth 2 and lower teeth 3 of a user and the rear end portion is extended backward along the tongue 6 of the user to press the tongue 6;
an upper teeth stopper projection 20 which is provided on the upper surface of the front end of the preventing tool body 10 and upwardly protrudes so as to prevent the upper teeth 2 of the user from protruding forward;
a lower teeth guide slope portion 30 which is provided on the bottom surface of the front end of the preventing tool body 10 and is upwardly inclined toward the front end so that, when the lower teeth 3 of the user bite the bottom surface of the preventing tool body 10, the lower teeth 3 can protrude forward further than the upper teeth 2 stopped by the upper teeth stopper projection 20;
a stopper wing 50 which is provided on the preventing tool body 10 and is expanded so as to be stopped by the lips 4, 5 of the user in front of the upper teeth stopper projection 20; wherein,
air paths 11, 12 of the shape of groove extending back and forth are formed on the sides of the preventing tool body 10 for the outer air to flow into the oral cavity of the user.

2. A snoring and sleep apnea preventing tool 1 to prevent snoring and sleep apnea of claim 1, wherein an insert hole 51 is penetrated on the stopper wing 50 in the back and forth direction, and the preventing tool body 10 is further includes a front inserting projection 40 extending forward from the front end of it and being releasably engaged into the insert hole 51 of the stopper wing 50, and the stopper wing 50 can be mountable on the front insert projection 40 while being moved back and forth along the front insert projection 40.
